(19) United States
(12) Patent Application Publication
(10) Pub. No.: US 2025/0031971 A1
(43) Pub. Date: Jan. 30, 2025

(54) METHODS AND SYSTEMS FOR IDENTIFYING LYMPH NODES USING FLUORESCENCE IMAGING DATA

(71) Applicant: Stryker Corporation, Kalamazoo, MI (US)

(72) Inventor: Lina GUREVICH, Vancouver (CA)

(73) Assignee: Stryker Corporation, Kalamazoo, MI (US)

(21) Appl. No.: 18/781,814

(22) Filed: Jul. 23, 2024

Related U.S. Application Data
(60) Provisional application No. 63/515,621, filed on Jul. 26, 2023.

(51) Int. Cl. A61B 5/00; A61B 90/00; G06T 7/00; G16H 50/70

(52) U.S. Cl. CPC A61B 5/0071; A61B 5/418; A61B 5/7246; A61B 5/7264; A61B 5/742; A61B 90/37; G06T 7/0012; G16H 50/70; A61B 2090/373; G06T 2207/10064; G06T 2207/30101

(57) ABSTRACT

The present disclosure relates generally to medical imaging, and more specifically to techniques for identifying at least one lymph node of a subject using fluorescence images. An exemplary method comprises obtaining a fluorescence image of a field of view including the at least one lymph node of the subject; obtaining a template image; comparing the template image with the fluorescence image to obtain one or more similarity values; and identifying at least one portion of the fluorescence image that corresponds to a location of the at least one lymph node based on the one or more similarity values.

### CROSS-REFERENCE TO RELATED APPLICATIONS

This application claims the benefit of U.S. Provisional Application No. 63/515,621 filed on Jul. 26, 2023, the entire content of which is incorporated herein by reference for all purposes.

### FIELD

The present disclosure relates generally to medical imaging, and more specifically to techniques for identifying at least one lymph node of a subject using fluorescence images.

### BACKGROUND

When a patient is given a diagnosis of cancer, one of the first questions to be answered is whether the cancer has spread from a primary site (e.g., breast, colon, lung) or if it is still contained in the detected area. The answer can have implications in the prognosis of the patient and the treatment prescribed. For the majority of solid tumors, the most powerful indicator of metastatic spread is the presence or absence of cancer cells in the regional lymph nodes.

Lymph nodes are part of the lymphatic system that is widely present throughout the body. The lymphatic system collects fluid that escapes from the cells, arteries, and veins, and returns the fluid, called lymph, back to the heart. The lymph nodes serve as filters in the lymphatic system to filter any foreign bodies from the lymph before it returns to the heart. As tumors secrete fluid and shed cells (i.e., metastasize), the lymphatic system is a likely reservoir for those fluids and cancer cells to deposit. Thus, if the lymph nodes in the area of the tumor can be localized, surgically removed, and evaluated to determine if there are any cancer cells trapped there, the extent of the spread of the cancer can be determined.

Sentinel lymph node biopsy is a method that involves localization of the lymph node that is the first in the lymph node chain to receive lymphatic drainage from a primary tumor site. This first draining node is referred to as the sentinel lymph node (SLN) and has been found to be a highly accurate indicator of the metastatic involvement of the entire lymph node basin in which it is situated. In short, if the sentinel node does not contain cancer, then the rest of the lymph nodes are also likely negative for cancer. Thus, instead of removing all of the lymph nodes in the lymphatic basin in the area of the tumor, only the sentinel node needs to be removed for further analysis.

Currently, the most common techniques for SLN mapping employed include blue dye staining and gamma imaging, which are frequently used together to improve the accuracy of the results.

The first technique involves the injection of a blue dye around the tumor site. The dye flows into the lymphatic basin, which receives lymphatic drainage from the tumor, and the nodes are stained blue. The surgeon can proceed to carefully dissect the nodal basin and visually identify blue-stained lymphatic vessels. These lymphatic vessels are followed to the SLN, the first blue node in the lymphatic chain from the tumor. The SLN is then excised, and its histologic status is determined by pathology. Although this method has been used successfully by several researchers, its success varies with the experience level of the surgeon and can result in a procedure that approaches the invasive level of a full dissection of the nodal basin.

The second technique uses a radiocolloid as the agent injected around the tumor. This radioactive tracer flows into the lymphatics in the same manner as the blue dye and deposits into the SLN. The advantage to this method is that a gamma detection probe is utilized to detect and mark the SLN at the skin pre-incision and intra-operatively. This method allows a small incision to be used, unlike the blue dye, when visual search for nodes is necessary. Once localized, the SLN is excised and its histologic status can be determined.

Recently, in addition to these two established procedures, the indocyanine green (ICG) fluorescence technique for the detection of SLN is found to be a valid and feasible method in clinical practice. This method, however, has limited efficacy in patients with a high BMI as lymph drainage channels and sentinel nodes cannot be adequately visualized due to high degree of signal absorption. Further, the lymph nodes in certain locations (e.g., armpit) are surrounded by layers of fat and are thus hard to visualize. As a result, a surgeon may need to dig through the tissue until a lymph node is visible to the naked eye. Another challenge with the ICG fluorescence technique is that it may be hard to tell from the fluorescence image whether a fluorescence signal is from a lymph node or another source in the body. The identification is done based on a surgeon's knowledge and is thus error-prone.

### SUMMARY

Disclosed herein are exemplary devices, apparatuses, systems, methods, and non-transitory storage media for automatically identifying one or more lymph nodes of a subject based on fluorescence images such as near-infrared (NIR) images. An exemplary system can perform template matching techniques, machine learning techniques, or any combination thereof. The systems, devices, and methods may be used in association with surgical procedures. Imaging and analysis may be performed pre-operatively, intra-operatively, post-operatively, and during diagnostic imaging sessions and procedures. For example, the system can automatically identify lymph nodes in images acquired in the course of a SLN mapping procedure (such as ICG fluorescence or scintigraphy) and enhance the visualization to allow physicians and surgeons to quickly and efficiently map the relevant sentinel nodes. For example, the system can automatically locate areas containing sentinel lymph nodes in NIR image frames, and these areas can be further visually enhanced to indicate both the shape and the depth of the located nodes.

An exemplary method for identifying at least one lymph node of a subject comprises: obtaining a fluorescence image of a field of view including the at least one lymph node of the subject; obtaining a template image; comparing the template image with the fluorescence image to obtain one or more similarity values; and identifying at least one portion of the fluorescence image that corresponds to a location of the at least one lymph node based on the one or more similarity values.

In some aspects, the method further comprises displaying the identified at least one portion of the fluorescence image.

In some aspects, displaying the identified at least one portion of the fluorescence image comprises displaying the fluorescence image and at least one bounding box indicative of the identified at least one portion of the fluorescence image.

In some aspects, the fluorescence image is displayed as part of an intraoperative video stream during a surgical procedure.

In some aspects, the surgical procedure comprises a sentinel lymph node mapping procedure.

In some aspects, the fluorescence image is a first fluorescence image, the field of view is a first field of view, the subject is a first subject, and the method further comprises: obtaining a second fluorescence image of a second field of view including a lymph node of a second subject different from the first subject; and cropping a portion of the second fluorescence image as the template image.

In some aspects, the cropped portion of the second fluorescence image depicts a center portion of the lymph node of the second subject.

In some aspects, the template image is generated based on a predefined intensity distribution pattern.

In some aspects, the template image is generated based on a plurality of candidate template images.

In some aspects, the plurality of candidate template images corresponds to a plurality of subjects.

In some aspects, comparing the template image with the fluorescence image comprises: comparing the template image with a plurality of patches of the fluorescence image; and generating a matrix of similarity values, each similarity value in the matrix indicative of a difference between the template image and a respective patch of the plurality of patches of the fluorescence image.

In some aspects, each similarity value in the matrix of similarity values is calculated based on a pixel-wise comparison between the template image and the respective patch of the plurality of patches of the fluorescence image.

In some aspects, the plurality of patches of the fluorescence image is a first plurality of patches of the fluorescence image and the method further comprises: resizing the fluorescence image; and comparing the template image with a second plurality of patches of the resized fluorescence image.

In some aspects, identifying the at least one portion of the fluorescence image corresponding to the at least one lymph node comprises: comparing each similarity value of the one or more similarity values with a predefined threshold.

In some aspects, the method further comprises performing contrast enhancement on the identified at least one portion of the fluorescence image to obtain an enhanced version of the identified at least one portion of the fluorescence image.

In some aspects, the method further comprises displaying the enhanced version of the identified at least one portion of the fluorescence image according to a color scheme.

In some aspects, the contrast enhancement comprises histogram equalization.

In some aspects, the template image is associated with a metastatic node, and the method further comprises determining whether the at least one lymph node is metastatic based on the one or more similarity values.

In some aspects, the method further comprises displaying a visual indication of the metastatic determination for each lymph node of the at least one lymph node.

In some aspects, the template image is generated based on a plurality of time-intensity curves.

In some aspects, the fluorescence image is generated based on a time series of signal intensity data.

An exemplary method for identifying at least one lymph node of a subject comprises: obtaining a fluorescence image of a field of view including the at least one lymph node of the subject; obtaining a template image; and identifying at least one portion of the fluorescence image that corresponds to a location of the at least one lymph node by inputting the template image and the fluorescence image into one or more trained neural networks.

In some aspects, the method further comprises displaying the identified at least one portion of the fluorescence image.

In some aspects, displaying the identified at least one portion of the fluorescence image comprises displaying the fluorescence image and at least one bounding box indicative of the identified at least one portion of the fluorescence image.

In some aspects, the fluorescence image is displayed as part of an intraoperative video stream during a surgical procedure.

In some aspects, the surgical procedure comprises a sentinel lymph node mapping procedure.

In some aspects, the fluorescence image is a first fluorescence image and the subject is a first subject, and the method further comprises: obtaining a second fluorescence image of a second field of view including a lymph node of a second subject different from the first subject; and cropping a portion of the second fluorescence image as the template image.

In some aspects, the cropped portion of the second fluorescence image depicts a center portion of the lymph node of the second subject.

In some aspects, the template image is generated based on a predefined intensity distribution pattern.

In some aspects, the template image is generated based on a plurality of candidate template images.

In some aspects, the plurality of candidate template images corresponds to a plurality of subjects.

In some aspects, the one or more trained neural networks comprise a Siamese network backbone.

In some aspects, the one or more trained neural networks comprise a cross-correlation module connected with the Siamese network backbone.

In some aspects, the one or more trained neural networks comprise a template localization subnetwork connected with the cross-correlation module.

In some aspects, the method further comprises performing contrast enhancement on the identified at least one portion of the fluorescence image to obtain an enhanced version of the identified at least one portion of the fluorescence image.

In some aspects, the method further comprises displaying the enhanced version of the identified at least one portion of the fluorescence image according to a color scheme.

In some aspects, the contrast enhancement comprises histogram equalization.

In some aspects, the one or more trained neural networks are configured to output a classification confidence value for each location of a plurality of locations in the fluorescence image and a template bounding box at the respective location.

In some aspects, the template image is associated with a metastatic node, and the method further comprises determining whether the at least one lymph node is metastatic based on an output of the one or more trained neural networks.

In some aspects, the method further comprises displaying a visual indication of the metastatic determination for each lymph node of the at least one lymph node.

In some aspects, the template image is generated based on a plurality of time-intensity curves.

In some aspects, the fluorescence image comprises a time series of signal intensity data.

An exemplary system for identifying at least one lymph node of a subject comprises: one or more processors; one or more memories; and one or more programs. The one or more programs are stored in the one or more memories and configured to be executed by the one or more processors. The one or more programs include instructions for: obtaining a fluorescence image of a field of view including the at least one lymph node of the subject; obtaining a template image; comparing the template image with the fluorescence image to obtain one or more similarity values; and identifying at least one portion of the fluorescence image that corresponds to a location of the at least one lymph node based on the one or more similarity values.

In some aspects, the one or more programs further include instructions for: displaying the identified at least one portion of the fluorescence image.

In some aspects, displaying the identified at least one portion of the fluorescence image comprises: displaying the fluorescence image and at least one bounding box indicative of the identified at least one portion of the fluorescence image.

In some aspects, the fluorescence image is displayed as part of an intraoperative video stream during a surgical procedure.

In some aspects, the surgical procedure comprises a sentinel lymph node mapping procedure.

In some aspects, the fluorescence image is a first fluorescence image, the field of view is a first field of view, the subject is a first subject, and the one or more programs further include instructions for: obtaining a second fluorescence image of a second field of view including a lymph node of a second subject different from the first subject; and cropping a portion of the second fluorescence image as the template image.

In some aspects, the cropped portion of the second fluorescence image depicts a center portion of the lymph node of the second subject.

In some aspects, the template image is generated based on a predefined intensity distribution pattern.

In some aspects, the template image is generated based on a plurality of candidate template images.

In some aspects, the plurality of candidate template images corresponds to a plurality of subjects.

In some aspects, comparing the template image with the fluorescence image comprises: comparing the template image with a plurality of patches of the fluorescence image; and generating a matrix of similarity values, each similarity value in the matrix indicative of a difference between the template image and a respective patch of the plurality of patches of the fluorescence image.

In some aspects, each similarity value in the matrix of similarity values is calculated based on a pixel-wise comparison between the template image and the respective patch of the plurality of patches of the fluorescence image.

In some aspects, the plurality of patches of the fluorescence image is a first plurality of patches of the fluorescence image, and the one or more programs further include instructions for: resizing the fluorescence image; and comparing the template image with a second plurality of patches of the resized fluorescence image.

In some aspects, identifying the at least one portion of the fluorescence image corresponding to the at least one lymph node comprises: comparing each similarity value of the one or more similarity values with a predefined threshold.

In some aspects, the one or more programs further include instructions for: performing contrast enhancement on the identified at least one portion of the fluorescence image to obtain an enhanced version of the identified at least one portion of the fluorescence image.

In some aspects, the one or more programs further include instructions for: displaying the enhanced version of the identified at least one portion of the fluorescence image according to a color scheme.

In some aspects, the contrast enhancement comprises histogram equalization.

In some aspects, the template image is associated with a metastatic node, and the one or more programs further include instructions for: determining whether the at least one lymph node is metastatic based on the one or more similarity values.

In some aspects, the one or more programs further include instructions for: displaying a visual indication of the metastatic determination for each lymph node of the at least one lymph node.

In some aspects, the template image is generated based on a plurality of time-intensity curves.

In some aspects, the fluorescence image is generated based on a time series of signal intensity data.

An exemplary system for identifying at least one lymph node of a subject comprises: one or more processors; one or more memories; and one or more programs. The one or more programs are stored in the one or more memories and configured to be executed by the one or more processors. The one or more programs include instructions for: obtaining a fluorescence image of a field of view including the at least one lymph node of the subject; obtaining a template image; and identifying at least one portion of the fluorescence image that corresponds to a location of the at least one lymph node by inputting the template image and the fluorescence image into one or more trained neural networks.

In some aspects, the one or more programs further include instructions for: displaying the identified at least one portion of the fluorescence image.

In some aspects, displaying the identified at least one portion of the fluorescence image comprises: displaying the fluorescence image and at least one bounding box indicative of the identified at least one portion of the fluorescence image.

In some aspects, the fluorescence image is displayed as part of an intraoperative video stream during a surgical procedure.

In some aspects, the surgical procedure comprises a sentinel lymph node mapping procedure.

In some aspects, the fluorescence image is a first fluorescence image, and the subject is a first subject, and the one or more programs further include instructions for: obtaining a second fluorescence image of a second field of view including a lymph node of a second subject different from the first subject; and cropping a portion of the second fluorescence image as the template image.

In some aspects, the cropped portion of the second fluorescence image depicts a center portion of the lymph node of the second subject.

In some aspects, the template image is generated based on a predefined intensity distribution pattern.

In some aspects, the template image is generated based on a plurality of candidate template images.

In some aspects, the plurality of candidate template images corresponds to a plurality of subjects.

In some aspects, the one or more trained neural networks comprise a Siamese network backbone.

In some aspects, the one or more trained neural networks comprise a cross-correlation module connected with the Siamese network backbone.

In some aspects, the one or more trained neural networks comprise a template localization subnetwork connected with the cross-correlation module.

In some aspects, the one or more programs further include instructions for: performing contrast enhancement on the identified at least one portion of the fluorescence image to obtain an enhanced version of the identified at least one portion of the fluorescence image.

In some aspects, the one or more programs further include instructions for: displaying the enhanced version of the identified at least one portion of the fluorescence image according to a color scheme.

In some aspects, the contrast enhancement comprises histogram equalization.

In some aspects any one of claims, the one or more trained neural networks are configured to output a classification confidence value for each location of a plurality of locations in the fluorescence image and a template bounding box at the respective location.

In some aspects, the template image is associated with a metastatic node, and the one or more programs further include instructions for: determining whether the at least one lymph node is metastatic based on an output of the one or more trained neural networks.

In some aspects, the one or more programs further include instructions for: displaying a visual indication of the metastatic determination for each lymph node of the at least one lymph node.

In some aspects, template image is generated based on a plurality of time-intensity curves.

In some aspects, the fluorescence image comprises a time series of signal intensity data.

Also disclosed herein is a kit for imaging tissue in a surgical site, the kit comprising a fluorescence imaging agent and any of the system described herein.

Also disclosed herein is use of the kit for lymphatic imaging.

Also disclosed herein is a fluorescence imaging agent for use with any of the surgical systems described herein for imaging tissue in a surgical site.

Optionally, imaging tissue in the surgical site comprises imaging lymphatic tissue.

Also disclosed herein is use of any of the system described herein for lymphatic imaging.

In some aspects, the method further comprises administering a fluorescence agent to the subject.

An exemplary non-transitory computer-readable storage medium stores one or more programs. The one or more programs comprise instructions, which when executed by one or more processors of an electronic device, cause the electronic device to perform any of methods described herein.

### BRIEF DESCRIPTION OF THE FIGURES

The patent or application file contains at least one drawing executed in color. Copies of this patent or patent application publication with color drawing(s) will be provided by the Office upon request and payment of the necessary fee.

The invention will now be described, by way of example only, with reference to the accompanying drawings, in which:

FIG. 1A is an illustration of an endoscopic camera system.

FIG. 1B is a diagram of a portion of the endoscopic camera system of FIG. 1A and a target object for imaging.

FIG. 2 illustrates a schematic view of a system for illumination and imaging.

FIG. 3 is a block diagram of an imaging system.

FIG. 4 depicts a graphical representation of a lymph node.

FIG. 5A depicts a white-light photo with an overlay of fluorescence signal (in green) depicting the skin of a pig after ICG injections.

FIG. 5B depicts a corresponding NIR image illustrating the ICG distribution in the tissue.

FIG. 5C depicts a plot of the pixel intensity distribution of the fluorescence image patch identified in the bounding box in FIG. 5B.

FIG. 5D depicts a three-dimensional graph of pixel intensities.

FIG. 6A includes an NIR image with a bounding box marking the location of an ICG injection site and a corresponding plot of the pixel intensity distribution of the image patch marked by the bounding box.

FIG. 6B includes an NIR image with a bounding box marking the location of a lymphatic channel and corresponding plot of the pixel intensity distribution of the image patch marked by the bounding box.

FIG. 6C includes an NIR image with a bounding box marking the location of a background noise region and a corresponding plot of the pixel intensity distribution of the image patch marked by the bounding box.

FIG. 7A includes an NIR image with a bounding box marking the location of an axillary lymph node of a human subject and a corresponding plot of the pixel intensity distribution of the image patch marked by the bounding box.

FIG. 7B includes an NIR image with a bounding box marking the location of an abdominal lymph node of a human subject and a corresponding plot of the pixel intensity distribution of the image patch marked by the bounding box.

FIG. 8 depicts a method for identifying at least one lymph node of a subject.

FIG. 9 depicts generation of a template image.

FIGS. 10A-10C depict exemplary identification of lymph nodes.

FIG. 11 depicts an exemplary identification of a lymph node with a dimmed fluorescence signal.

FIG. 12A depicts a visualization of a similarity matrix for a fluorescence image.

FIG. 12B depicts an enhanced visualization of an identified lymph node in the fluorescence image of FIG. 12A.

FIG. 13 depicts a diagram of a method for identifying at least one lymph node of a subject.

FIG. 14 depicts an exemplary neural network model.

FIG. 15A depicts a graphical representation of a ureter vascular system.

FIG. 15B depicts the appearance of a ureter vascular system under fluorescence.

### DETAILED DESCRIPTION

Reference will now be made in detail to implementations and various aspects and variations of systems and methods described herein. Although several exemplary variations of the systems and methods are described herein, other variations of the systems and methods may include aspects of the systems and methods described herein combined in any suitable manner having combinations of all or some of the aspects described. Examples will now be described more fully hereinafter with reference to the accompanying drawings; however, they may be embodied in different forms and should not be construed as limited to the examples set forth herein. Rather, these examples are provided so that this disclosure will be thorough and complete, and will fully convey exemplary implementations to those skilled in the art.

Disclosed herein are exemplary devices, apparatuses, systems, methods, and non-transitory storage media for automatically identifying one or more lymph nodes of a subject based on fluorescence images such as near-infrared (NIR) images. An exemplary system can perform template matching techniques, machine learning techniques, or any combination thereof. The systems, devices, and methods may be used in association with surgical procedures. Imaging and analysis may be performed pre-operatively, intra-operatively, post-operatively, and during diagnostic imaging sessions and procedures. For example, the system can automatically identify lymph nodes in images acquired in the course of a SLN mapping procedure (such as ICG fluorescence or scintigraphy) and enhance the visualization to allow physicians and surgeons to quickly and efficiently map the relevant sentinel nodes. For example, the system can automatically locate areas containing sentinel lymph nodes in NIR image frames, and these areas can be further visually enhanced to indicate both the shape and the depth of the located nodes.

The system can obtain a fluorescence image of a field of view including the at least one lymph node of the subject and a template image. For a given imaging modality (i.e., ICG fluorescence), certain spatial intensity distribution patterns are unique to sentinel lymph nodes and are unlikely to be produced by other types of tissue, noise, or background. As described herein, in the context of fluorescence imaging, an area of fluorescence with a mountain-like intensity distribution pattern is highly likely to correspond to a sentinel lymph node. Accordingly, the template image can be generated by excising or cropping a relevant image patch from a fluorescence image where the location of the tissue of interest (e.g., lymph node) is known and/or be artificially modelled based on an identified signature pattern of a tissue of interest (e.g., lymph node).

The system can execute a template matching algorithm to compare the fluorescence image and the template image. Based on the comparison, the system can obtain a matrix of similarity values. The system can then compare the matrix with a predefined threshold to identify the most probable locations of the lymph node(s). The locations within the fluorescence image with similarity scores over the predefined threshold are marked as the areas containing lymph node(s). To account for differences in scale between the input image and the template, the system can resize the input image in successive runs of the template matching process, as described herein.

Additionally or alternatively, the system can execute one or more machine learning algorithms to identify lymph nodes. For example, the system can identify at least one portion of the fluorescence image that corresponds to a location of the at least one lymph node by inputting the template image and the fluorescence image into one or more trained neural networks, as described herein.

After the lymph node(s) are identified, the system can locally enhance the region(s) corresponding to the lymph node(s) by, for example, contrast enhancement (e.g., histogram equalization). The system can further display the region(s) according to a color scheme to convey the depth of the node. If multiple fluorescence images are obtained from a video, the timing component can also be incorporated into the node visualization. For example, the nodes that are identified earlier in the course of the imaging process can be shown differently from the ones that are identified later, because the nodes that absorb the dye sooner are more likely to be the primary sentinel nodes.

Examples of the present disclosure provide numerous technical advantages. As discussed herein, the ICG fluorescence technique for the detection of SLN has limited efficacy in patients with a high BMI as lymph drainage channels and sentinel nodes cannot be adequately visualized due to high degree of signal absorption. Further, the lymph nodes in certain locations (e.g., armpit) are surrounded by layers of fat and are thus hard to visualize. As a result, a surgeon may need to dig through the tissue until a lymph node is visible to the naked eye. Another challenge with the ICG fluorescence technique is that it may be hard to tell from the fluorescence image whether a fluorescence signal is from a lymph node or another source in the body. The identification is done based on a surgeon's knowledge and is thus error-prone. Techniques described herein can accurately identify lymph nodes based on a distinct pattern even if the fluorescence signal is weak and/or invisible to manual identification. Further, the techniques described herein can also determine the likelihood that the node is metastatic or benign. Further still, the techniques described herein can also be used to identify any tissue/structure of interest exhibiting a distinct visual and/or temporal pattern, as described herein.

In the following description, it is to be understood that the singular forms “a,” “an,” and “the” used in the following description are intended to include the plural forms as well, unless the context clearly indicates otherwise. It is also to be understood that the term “and/or” as used herein refers to and encompasses any and all possible combinations of one or more of the associated listed items. It is further to be understood that the terms “includes, “including,” “comprises,” and/or “comprising,” when used herein, specify the presence of stated features, integers, steps, operations, elements, components, and/or units but do not preclude the presence or addition of one or more other features, integers, steps, operations, elements, components, units, and/or groups thereof.

Certain aspects of the present disclosure include process steps and instructions described herein in the form of an algorithm. It should be noted that the process steps and instructions of the present disclosure could be embodied in software, firmware, or hardware and, when embodied in software, could be downloaded to reside on and be operated from different platforms used by a variety of operating systems. Unless specifically stated otherwise as apparent from the following discussion, it is appreciated that, throughout the description, discussions utilizing terms such as “processing,” “computing,” “calculating,” “determining,” “displaying,” “generating” or the like, refer to the action and processes of a computer system, or similar electronic computing device, that manipulates and transforms data represented as physical (electronic) quantities within the computer system memories or registers or other such information storage, transmission, or display devices.

The present disclosure in some examples also relates to a device for performing the operations herein. This device may be specially constructed for the required purposes, or it may comprise a general purpose computer selectively activated or reconfigured by a computer program stored in the computer. Such a computer program may be stored in a non-transitory, computer readable storage medium, such as, but not limited to, any type of disk, including floppy disks, USB flash drives, external hard drives, optical disks, CD-ROMs, magnetic-optical disks, read-only memories (ROMs), random access memories (RAMs), EPROMs, EEPROMs, magnetic or optical cards, application specific integrated circuits (ASICs), or any type of media suitable for storing electronic instructions, and each coupled to a computer system bus. Furthermore, the computers referred to in the specification may include a single processor or may be architectures employing multiple processor designs for increased computing capability.

The methods, devices, and systems described herein are not inherently related to any particular computer or other apparatus. Various general-purpose systems may also be used with programs in accordance with the teachings herein, or it may prove convenient to construct a more specialized apparatus to perform the required method steps. The required structure for a variety of these systems will appear from the description below. In addition, the present invention is not described with reference to any particular programming language. It will be appreciated that a variety of programming languages may be used to implement the teachings of the present invention as described herein.

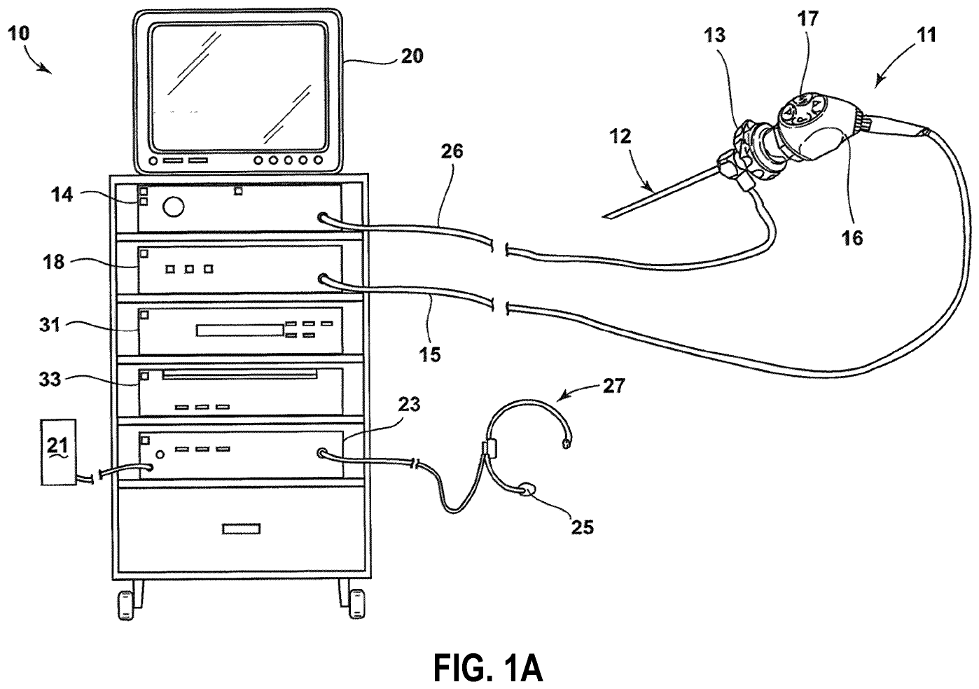

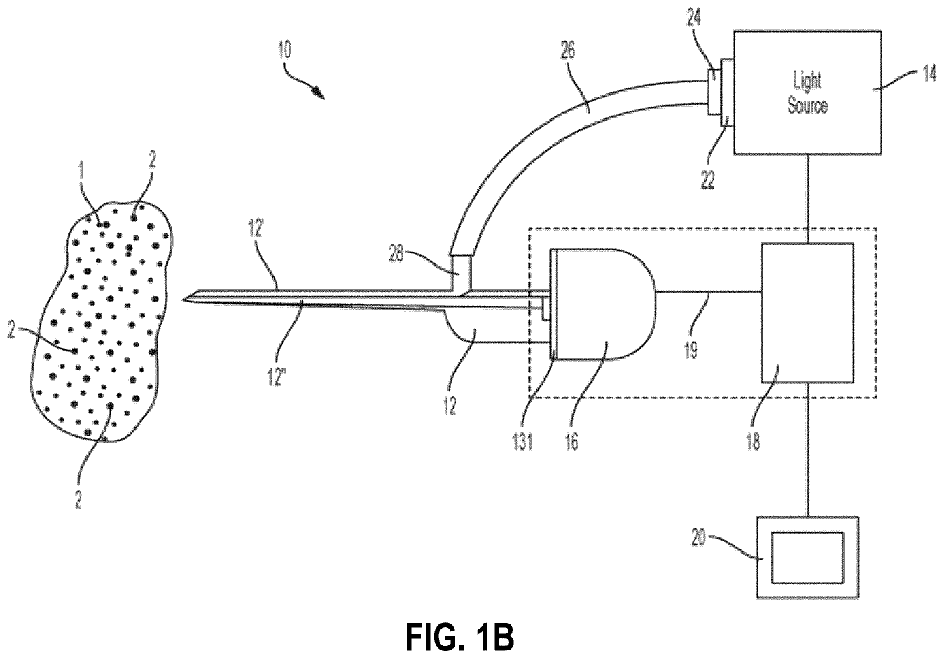

FIG. 1A shows an example of an endoscopic imaging system 10, which includes a scope assembly 11 which may be utilized in endoscopic procedures. The scope assembly 11 incorporates an endoscope or scope 12 which is coupled to a camera head 16 by a coupler 13 located at the distal end of the camera head 16. Light is provided to the scope by a light source 14 via a light guide 26, such as a fiber optic cable. The camera head 16 is coupled to a camera control unit (CCU) 18 by an electrical cable 15. The CCU 18 is connected to, and communicates with, the light source 14. Operation of the camera 16 is controlled, in part, by the CCU 18. The cable 15 conveys video image and/or still image data from the camera head 16 to the CCU 18 and may convey various control signals bi-directionally between the camera head 16 and the CCU 18.

A control or switch arrangement 17 may be provided on the camera head 16 for allowing a user to manually control various functions of the system 10, which may include switching from one imaging mode to another, as discussed further below. Voice commands may be input into a microphone 25 mounted on a headset 27 worn by the practitioner and coupled to the voice-control unit 23. A hand-held control device 21, such as a tablet with a touch screen user interface or a PDA, may be coupled to the voice control unit 23 as a further control interface. In the illustrated example, a recorder 31 and a printer 33 are also coupled to the CCU 18. Additional devices, such as an image capture and archiving device, may be included in the system 10 and coupled to the CCU 18. Video image data acquired by the camera head 16 and processed by the CCU 18 is converted to images, which can be displayed on a monitor 20, recorded by recorder 31, and/or used to generate static images, hard copies of which can be produced by the printer 33.

FIG. 1B shows an example of a portion of the endoscopic system 10 being used to illuminate and receive light from an object 1, such as a surgical site of a patient. The object 1 may include fluorescent markers 2, for example, as a result of the patient being administered a fluorescence imaging agent. The fluorescent markers 2 may comprise, for example, indocyanine green (ICG).

The light source 14 can generate visible illumination light (such as any combination of red, green, and blue light) for generating visible (e.g., white light) images of the target object 1 and, in some examples, can also produce fluorescence excitation illumination light for exciting the fluorescent markers 2 in the target object for generating fluorescence images. In some examples, the light source 14 can produce fluorescence excitation illumination light for exciting autofluorescence in the target object for generating fluorescence images, additionally or alternatively to light for exciting the fluorescent markers. Illumination light is transmitted to and through an optic lens system 22 which focuses light onto a light pipe 24. The light pipe 24 may create a homogeneous light, which is then transmitted to the fiber optic light guide 26. The light guide 26 may include multiple optic fibers and is connected to a light post 28, which is part of the endoscope 12. The endoscope 12 includes an illumination pathway 12′ and an optical channel pathway 12″.

The endoscope 12 may include a notch filter 131 that allows some or all (preferably, at least 80%) of fluorescence emission light (e.g., in a wavelength range of 830 nm to 870 nm) emitted by fluorescence markers 2 in the target object 1 to pass therethrough and that allows some or all (preferably, at least 80%) of visible light (e.g., in the wavelength range of 400 nm to 700 nm), such as visible illumination light reflected by the target object 1, to pass therethrough, but that blocks substantially all of the fluorescence excitation light (e.g., infrared light having a wavelength of 808 nm) that is used to excite fluorescence emission from the fluorescent marker 2 in the target object 1. The notch filter 131 may have an optical density of OD5 or higher. In some examples, the notch filter 131 can be located in the coupler 13.

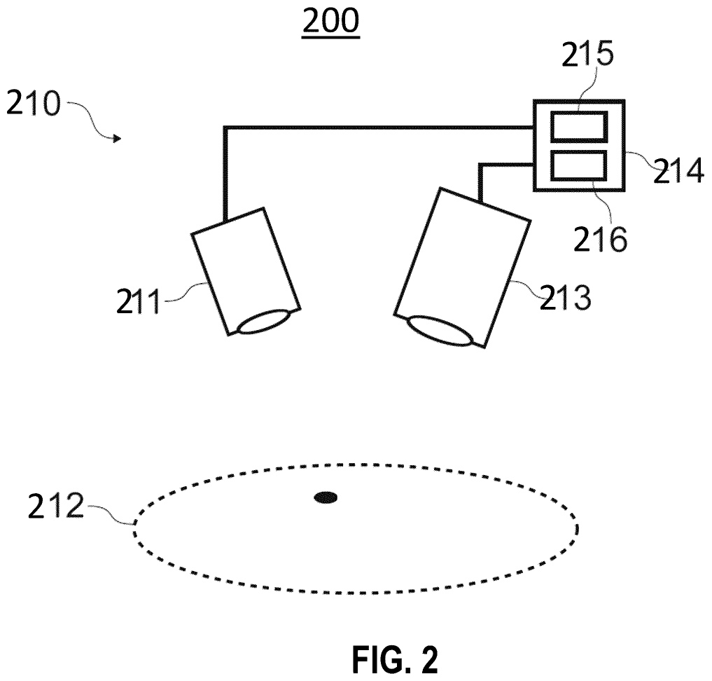

FIG. 2 illustrates an exemplary open field imaging system in accordance with some examples. FIG. 2 illustrates a schematic view of an illumination and imaging system 210 that can be used in open field surgical procedures. As may be seen therein, the system 210 may include an illumination module 211, an imaging module 213, and a video processor/illuminator (VPI) 214. The VPI 214 may include an illumination source 215 to provide illumination to the illumination module 211 and a processor assembly 216 to send control signals and to receive data about light detected by the imaging module 213 from a target 212 illuminated by light output by the illumination module 211. In one variation, the video processor/illuminator 214 may comprise a separately housed illumination source 215 and the processor assembly 216. In one variation, the video processor/illuminator 214 may comprise the processor assembly 216 while one or more illumination sources 215 are separately contained within the housing of the illumination module 211. The illumination source 215 may output light at different waveband regions, e.g., white (RGB) light, excitation light to induce fluorescence in the target 212, a combination thereof, and so forth, depending on characteristics to be examined and the material of the target 212. Light at different wavebands may be output by the illumination source 215 simultaneously, sequentially, or both. The illumination and imaging system 210 may be used, for example, to facilitate medical (e.g., surgical) decision making, e.g., during a surgical procedure. The target 212 may be a topographically complex target, e.g., a biological material including tissue, an anatomical structure, other objects with contours and shapes resulting in shadowing when illuminated, and so forth. The VPI 214 may record, process, display, and so forth, the resulting images and associated information.

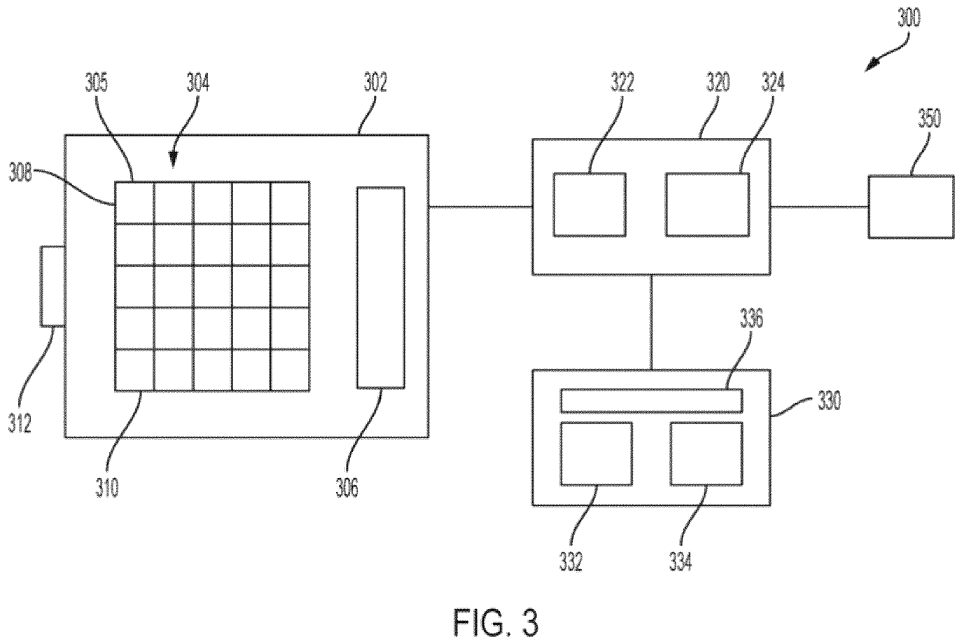

FIG. 3 schematically illustrates an exemplary imaging system 300 that employs an electronic imager 302 to generate images (e.g., still and/or video) of a target object, such as a target tissue of a patient, according to some examples. The imager 302 may be a rolling shutter imager (e.g., CMOS sensors) or a global shutter imager (e.g., CCD sensors). System 300 may be used, for example, for the endoscopic imaging system 10 of FIG. 1A. The imager 302 includes a CMOS sensor 304 having an array of pixels 305 arranged in rows of pixels 308 and columns of pixels 310. The imager 302 may include control components 306 that control the signals generated by the CMOS sensor 304. Examples of control components include gain circuitry for generating a multi-bit signal indicative of light incident on each pixel of the sensor 304, one or more analog-to-digital converters, one or more line drivers to act as a buffer and provide driving power for the sensor 304, row circuitry, and timing circuitry. A timing circuit may include components such as a bias circuit, a clock/timing generation circuit, and/or an oscillator. Row circuitry may enable one or more processing and/or operational tasks such as addressing rows of pixels 308, addressing columns of pixels 310, resetting charge on rows of pixels 308, enabling exposure of pixels 305, decoding signals, amplifying signals, analog-to-digital signal conversion, applying timing, read out and reset signals, and other suitable processes or tasks. Imager 302 may also include a mechanical shutter 312 that may be used, for example, to control exposure of the image sensor 304 and/or to control an amount of light received at the image sensor 304.

One or more control components may be integrated into the same integrated circuit in which the sensor 304 is integrated or may be discrete components. The imager 302 may be incorporated into an imaging head, such as camera head 16 of system 10.

One or more control components 306, such as row circuitry and a timing circuit, may be electrically connected to an imaging controller 320, such as camera control unit 18 of system 10. The imaging controller 320 may include one or more processors 322 and memory 324. The imaging controller 320 receives imager row readouts and may control readout timings and other imager operations, including mechanical shutter operation. The imaging controller 320 may generate image frames, such as video frames from the row and/or column readouts from the imager 302. Generated frames may be provided to a display 350 for display to a user, such as a surgeon.

The system 300 in this example includes a light source 330 for illuminating a target scene. The light source 330 is controlled by the imaging controller 320. The imaging controller 320 may determine the type of illumination provided by the light source 330 (e.g., white light, fluorescence excitation light, or both), the intensity of the illumination provided by the light source 330, and or the on/off times of illumination in synchronization with rolling shutter operation. The light source 330 may include a first light generator 332 for generating light in a first wavelength and a second light generator 334 for generating light in a second wavelength. In some examples, the first light generator 332 is a white light generator, which may be comprised of multiple discrete light generation components (e.g., multiple LEDs of different colors), and the second light generator 334 is a fluorescence excitation light generator, such as a laser diode.

The light source 330 includes a controller 336 for controlling light output of the light generators. The controller 336 may be configured to provide pulse width modulation (PWM) of the light generators for modulating intensity of light provided by the light source 330, which can be used to manage over-exposure and under-exposure. In some examples, nominal current and/or voltage of each light generator remains constant, and the light intensity is modulated by switching the light generators (e.g., LEDs) on and off according to a PWM control signal. In some examples, a PWM control signal is provided by the imaging controller 336. This control signal can be a waveform that corresponds to the desired pulse width modulated operation of light generators.

The imaging controller 320 may be configured to determine the illumination intensity required of the light source 330 and may generate a PWM signal that is communicated to the light source 330. In some examples, depending on the amount of light received at the sensor 304 and the integration times, the light source may be pulsed at different rates to alter the intensity of illumination light at the target scene. The imaging controller 320 may determine a required illumination light intensity for a subsequent frame based on an amount of light received at the sensor 304 in a current frame and/or one or more previous frames. In some examples, the imaging controller 320 is capable of controlling pixel intensities via PWM of the light source 330 (to increase/decrease the amount of light at the pixels), via operation of the mechanical shutter 312 (to increase/decrease the amount of light at the pixels), and/or via changes in gain (to increase/decrease sensitivity of the pixels to received light). In some examples, the imaging controller 320 primarily uses PWM of the illumination source for controlling pixel intensities while holding the shutter open (or at least not operating the shutter) and maintaining gain levels. The controller 320 may operate the shutter 312 and/or modify the gain in the event that the light intensity is at a maximum or minimum and further adjustment is needed.

### Morphology of a Lymph Node

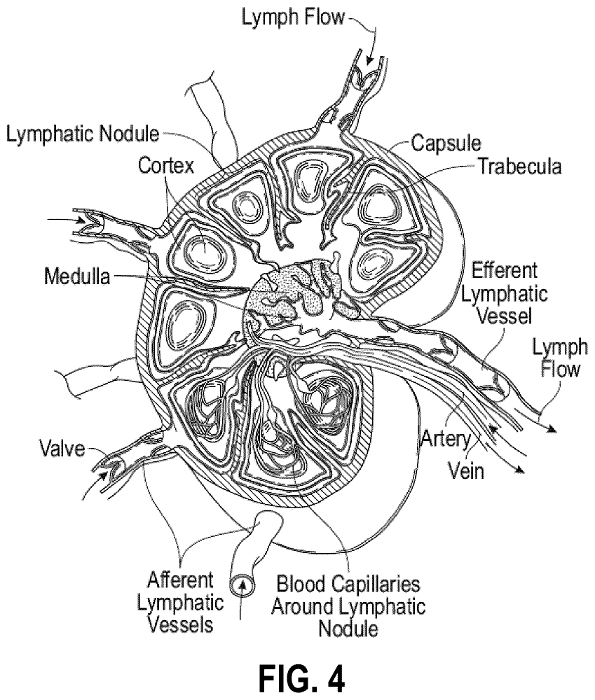

Examples of the present disclosure can identify one or more lymph nodes of a subject in an image based on the unique morphology of a typical lymph node. FIG. 4 provides a graphical representation of a lymph node. As shown, the lymph node is an ovoid or kidney-shaped organ. Lymph nodes are a part of the lymphatic system that is widely present throughout the human body and are linked by lymphatic vessels as a part of the circulatory system. When a fluorescence imaging agent is absorbed by lymphatic channels and delivered to the sentinel lymph nodes, the lymph nodes can be visualized via exposure to a fluorescence excitation light source. Due to the characteristic ovoid shape of each node as shown in FIG. 4, when the collected fluorescence signal is rendered in the two-dimensional space of an image, the spatial distribution of pixel intensities within the area of the node follows a signature pattern reflecting the curvature of the node, as further shown herein. Specifically, the points on the surface of the node, which are closest to the excitation source, can appear brighter than the points further down from the peak. Furthermore, due to the symmetric properties of an ovoid shape, this intensity distribution pattern can demonstrate rotational symmetry relative to the axis originating at the point of peak intensity and orthogonal to the surface of the image.

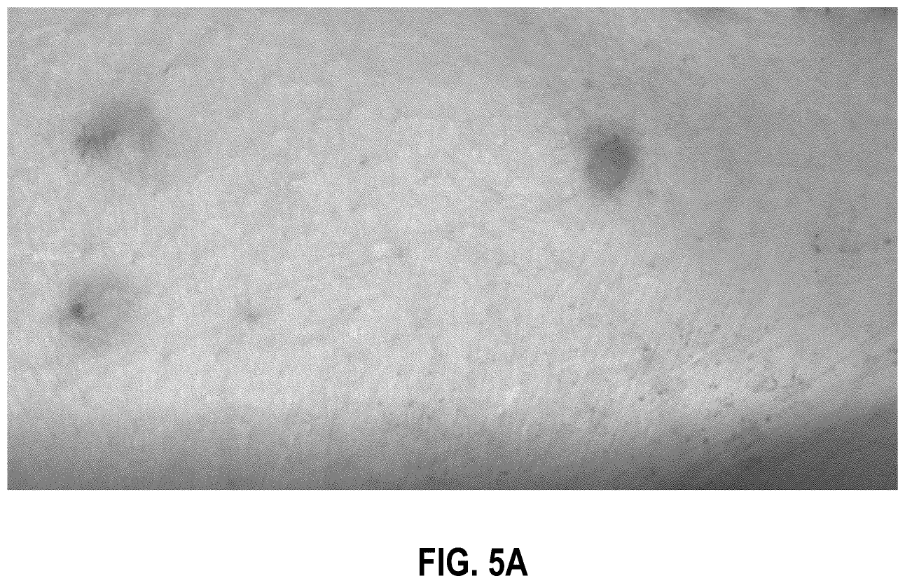

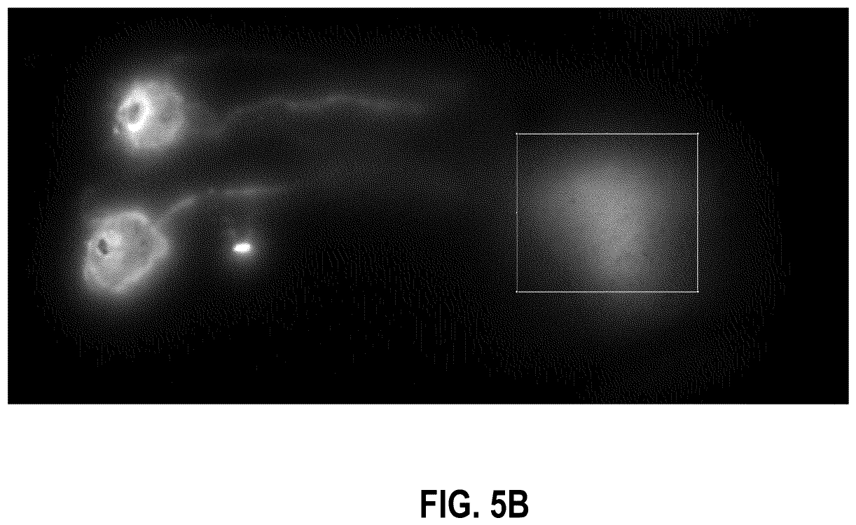

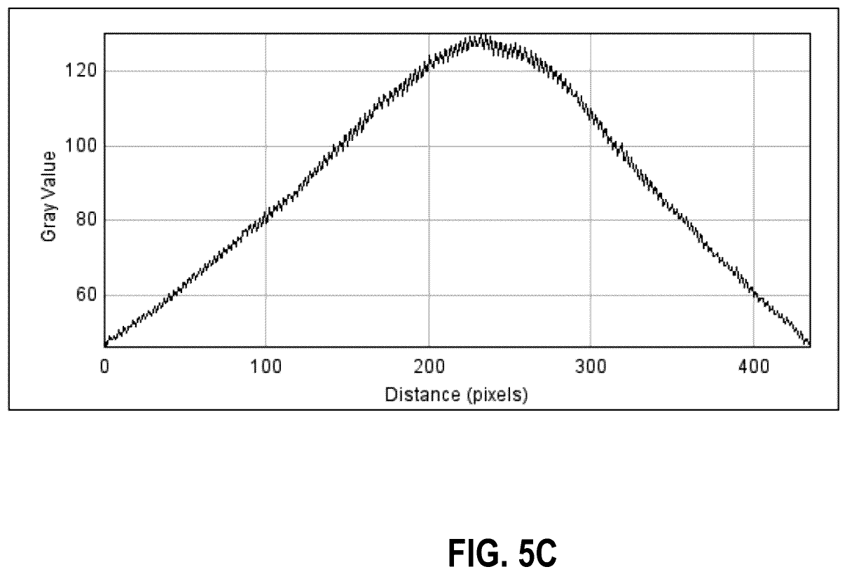

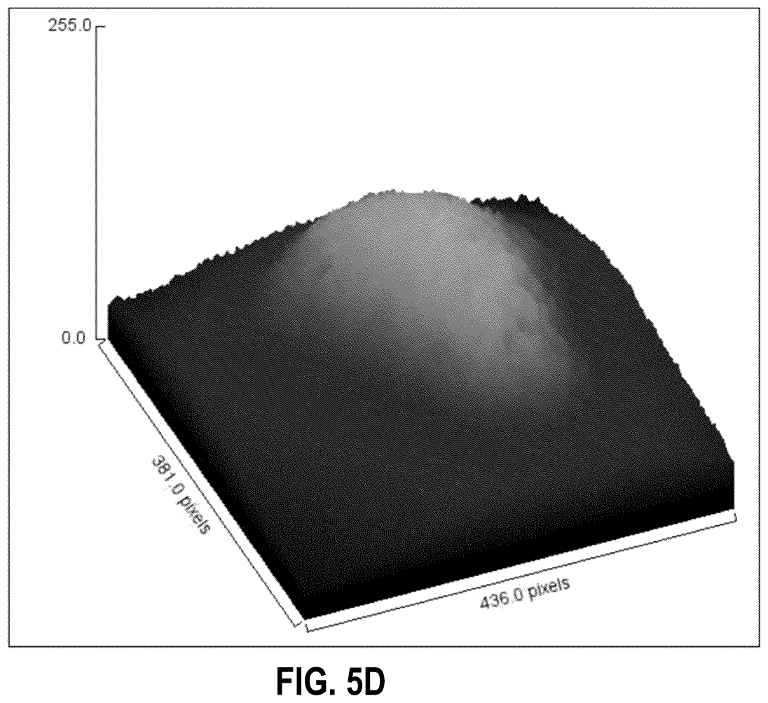

FIGS. 5A-5D demonstrate how the unique morphology of a lymph node can result in a specific signal intensity pattern in fluorescence images. FIG. 5A depicts a white-light photo with an overlay of fluorescence signal (in green) depicting the skin of a pig after ICG injections. In FIG. 5A, the two bright green areas on the left mark the ICG injection sites and an ovoid shape in dimmer green on the right marks a subdermal lymph node. FIG. 5B depicts a corresponding NIR image illustrating the ICG distribution in the tissue. Similarly to FIG. 5A, FIG. 5B shows two bright spots on the left marking the injection sites and an ovoid shape on the right marking the subdermal lymph node. The two lines originating from the two injection sites correspond to the propagation of the dye through the lymphatic channels to the lymph node. In FIG. 5B, the location of the lymph node is marked in a bounding box.

FIG. 5C depicts a plot of the pixel intensity distribution of the fluorescence image patch identified in the bounding box in FIG. 5B. FIG. 5C is a column average plot, where the x-axis of the plot represents different x-axis locations in the fluorescence image patch, and the y-axis of the plot represents the vertically averaged pixel intensity at a corresponding x-axis location in the fluorescence image patch. As shown, the pixel intensity distribution follows a distinct mountain-shaped pattern. Similarly, FIG. 5D depicts a three-dimensional graph of pixel intensities and also follows a distinct mountain-shaped pattern. Accordingly, the unique morphology of a lymph node results in a distinct visual signature of the corresponding fluorescence signal.

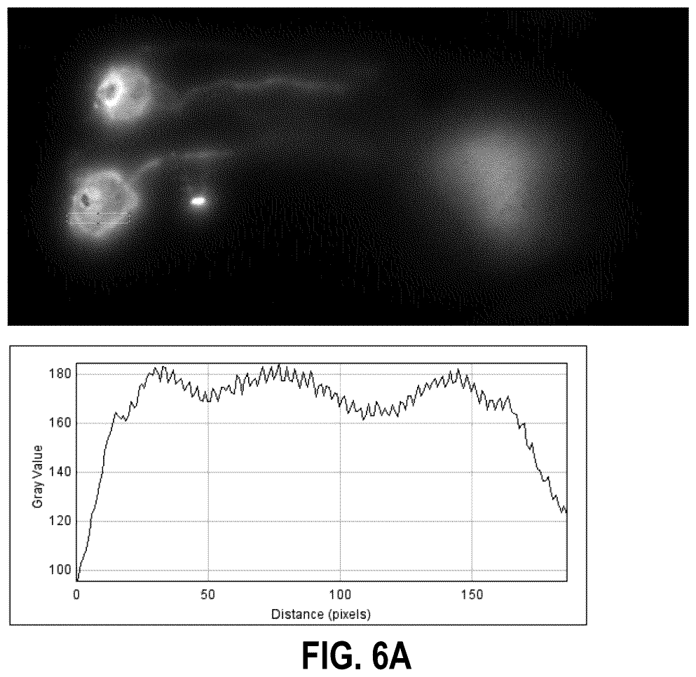

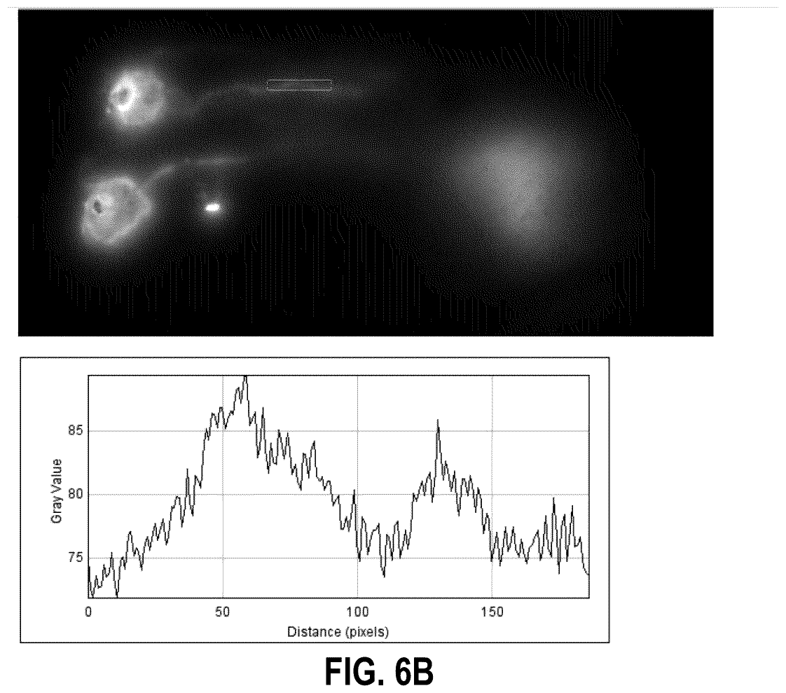

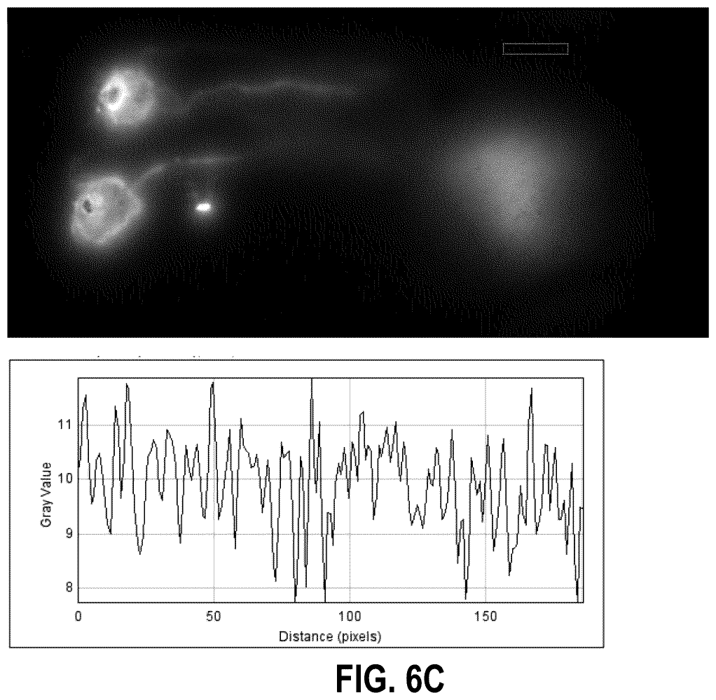

FIGS. 6A-C demonstrate that the mountain-shaped pattern is unique to lymph nodes. Specifically, FIGS. 6A-C depict spatial intensity distributions for various locations in the fluorescence image that do not enclose a lymph node. FIG. 6A depicts a plot of the pixel intensity distribution of an image patch corresponding to the injection site, which is identified in the bounding box in the fluorescence image. FIG. 6B depicts a plot of the pixel intensity distribution of an image patch corresponding to a lymphatic channel, which is identified in the bounding box in the fluorescence image. FIG. 6C depicts a plot of the pixel intensity distribution of an image patch corresponding to a background noise region, which is identified in the bounding box in the fluorescence image. As shown, none of the non-lymphatic areas of the fluorescence image show the distinct mountain-shaped distribution pattern.

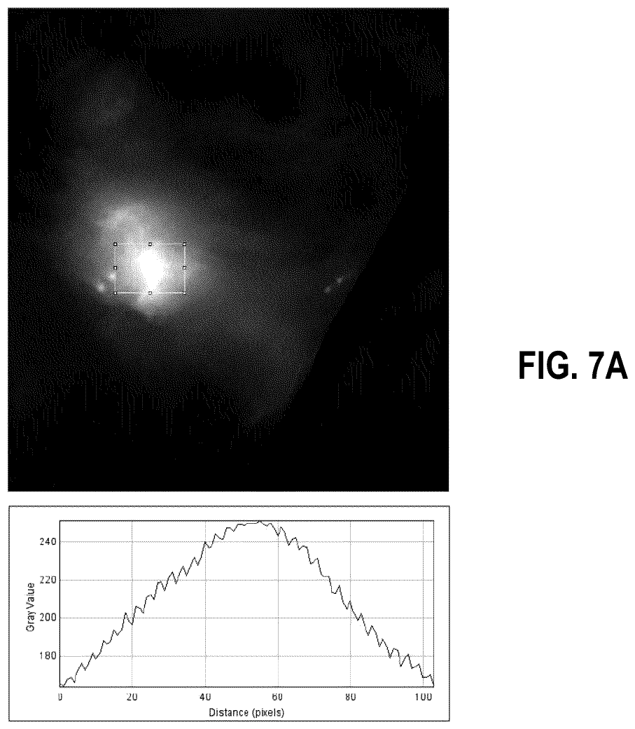

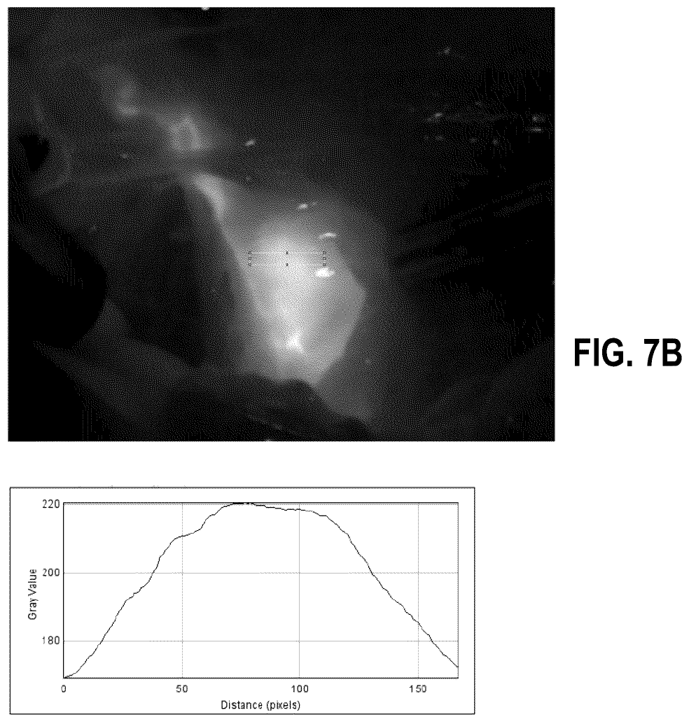

FIGS. 7A-B demonstrate that lymph nodes in different anatomies also produce the same distinct visual pattern. FIG. 7A includes an NIR image with a bounding box marking the location of an exposed axillary lymph node of a human subject, as well as a corresponding plot of the pixel intensity distribution of the image patch marked by the bounding box. FIG. 7B includes an NIR image with a bounding box marking the location of an abdominal lymph node of a human subject, as well as a corresponding plot of the pixel intensity distribution of the image patch marked by the bounding box. Both figures demonstrate that the lymph nodes produce the distinct mountain-shaped visual pattern.

### Techniques for Identifying Lymph Nodes

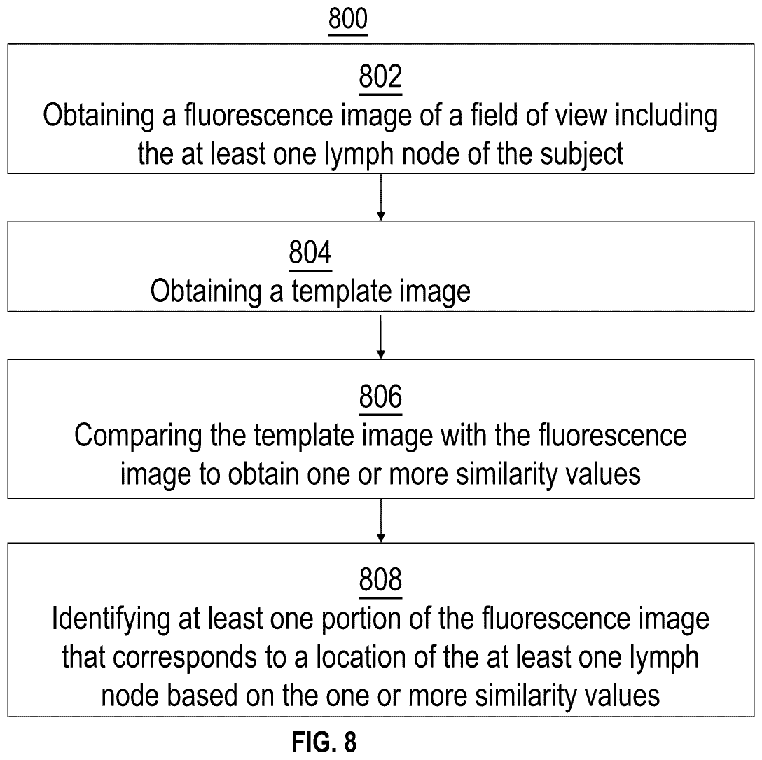

FIG. 8 illustrates an exemplary method 800 for identifying at least one lymph node of a subject. The method 800 can automatically recognize the distinct signature of the lymph node intensity distribution in a given NIR input image. Process 800 is performed, for example, using one or more electronic devices implementing a software platform. In some examples, process 800 is performed using a client-server system, and the blocks of process 800 are divided up in any manner between the server and one or more client devices. In some examples, process 800 is performed using only a client device or only multiple client devices. In process 800, some blocks are, optionally, combined, the order of some blocks is, optionally, changed, and some blocks are, optionally, omitted. In some examples, additional steps may be performed in combination with the process 800. Accordingly, the operations as illustrated (and described in greater detail below) are exemplary by nature and, as such, should not be viewed as limiting.

At block 802, an exemplary system (e.g., one or more electronic devices) obtains a fluorescence image of a field of view including the at least one lymph node of the subject. The fluorescence image may be captured before, during, and/or after a surgical procedure.

At block 804, the system obtains a template image. The template image is the image to which the fluorescence image is to be compared against to identify the at least one lymph node. The generation of the template image can be based on one or more other images or a predefined intensity distribution pattern (e.g., one that follows a mountain-shaped intensity distribution pattern). The generation of the template image can occur before the step 804.

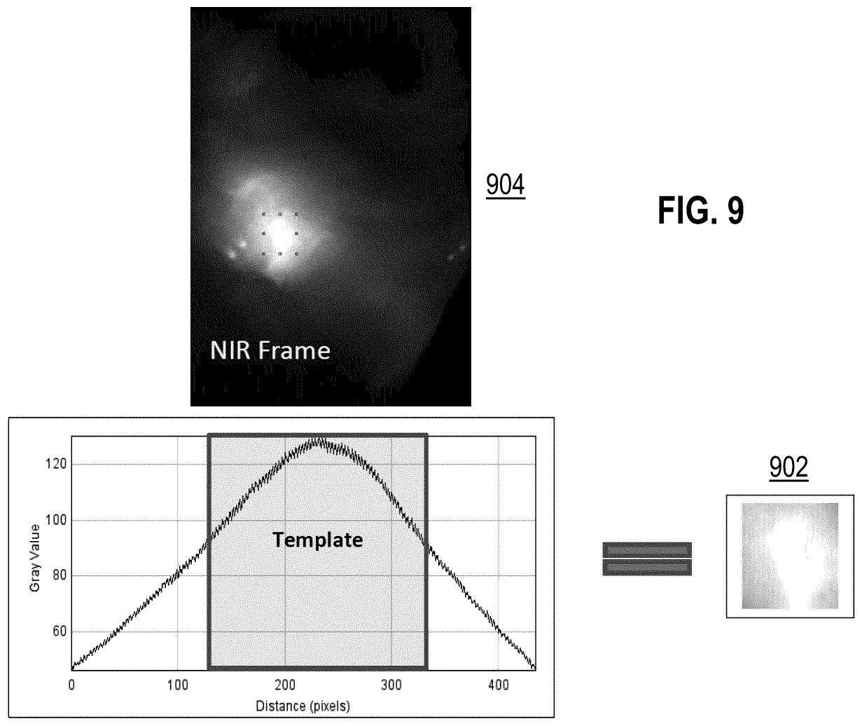

The generation of the template image can be based on a second fluorescence image different from the fluorescence image in block 802, and the second fluorescence image can be of a second subject different from the subject in block 802. The second subject may be a human or an animal (e.g., porcine). To generate the template image, the system can obtain the second fluorescence image having a field of view including a lymph node of the second subject and crop a portion of the second fluorescence image as the template image. The cropped portion of the second fluorescence image may depict a center portion of the lymph node of the second subject. In some examples, the template image is generated based on a plurality of candidate template images, which may correspond to a plurality of subjects. An exemplary template image is shown in FIG. 9.

At block 806, the system compares the template image with the fluorescence image to obtain one or more similarity values. The comparison may be performed using template matching techniques, which can involve searching and finding the location of a template image in a larger image (i.e., the input fluorescence image). For example, the system can slide the template image over the input image and compare the template image and the patch of input image under the template image. Specifically, to perform the comparison, the system can compare the template image with a plurality of patches of the fluorescence image and generating a matrix of similarity values, each similarity value in the matrix indicative of a difference between the template image and a respective patch of the plurality of patches of the fluorescence image. Each similarity value in the matrix of similarity values is calculated based on a pixel-wise comparison between the template image and the respective patch of the plurality of patches of the fluorescence image. For example, a location in the similarity matrix may correspond to a pixel location in the fluorescence image, and the similarity value at the location in the similarity matrix indicates the level of similarity between the template image and an image patch of the fluorescence image in which the pixel is at the center of the image patch.

In one exemplary implementation, the system receives a source image I, which is the fluorescence image obtained in block 802, and a template image T obtained in block 804. For each location of T over I, a Result Metric (R) is calculated using a matching method. It should be appreciated by one of ordinary skill in the art that many matching methods can be used, such as cross-correlation and sum of absolute differences. In the exemplary implementation, the following formula for R can be used.

$$R ( x , y ) = \frac{{\sum }_{{x}^{′} , {y}^{′}} ( {T}^{′} ( {x}^{′} , {y}^{′} ) \cdot {I}^{′} ( x + {x}^{′} , y + {y}^{′} ) )}{\sqrt{{\sum }_{{x}^{′} , {y}^{′}} T {′ ( {x}^{′} , {y}^{′} )}^{2} \cdot {\sum }_{{x}^{′} , y ′} I {′ ( x + {x}^{′} , y + {y}^{′} )}^{2}}}$$

$${T}^{′} ( {x}^{′} , {y}^{′} ) = T ( {x}^{′} , {y}^{′} ) - I / ( w \cdot h ) \cdot {\sum }_{{x}^{′′} y ′′} T ( {x}^{′′} , {y}^{′′} )$$

$${I}^{′} ( x + {x}^{′} , y + {y}^{′} ) = I ( x + {x}^{′} , y + {y}^{′} ) - I / ( w \cdot h ) \cdot {\sum }_{{x}^{′′} y ′′} I ( x + {x}^{′′} , y + {y}^{′′} )$$

Where I denotes the source image of size W×H and T denotes the template image of size w×h.
   The summation is performed over the template and/or the image patch: x′=0 . . . w−1, y′=0 . . . h−1

The higher the R score (i.e., the similarity score), the more similar the examined image patch is to the template image. Optionally, the similarity matrix may be normalized based on normalized cross correlation such that each similarity value in the matrix is in the range of zero to one.

At block 808, the system identifies at least one portion of the fluorescence image that corresponds to a location of the at least one lymph node based on the one or more similarity values. The system can examine the similarity matrix and identify one or more similarity values in the similarity matrix that exceed a predefined threshold. The predefined threshold is indicative of the minimum similarity score to be identified as a lymph node. Based on the one or more similarity values, the system can then identify one or more corresponding portions or patches of the fluorescence image (i.e., the image patch that, when compared against the template image, has produced the similarity score). The predefined threshold may optionally be empirically defined.

To improve the accuracy of the result, the system may use multiple templates and/or different orientations of the same template. Further, to address the issue of different scales between the input fluorescence image and the template, the system may execute the template matching algorithm repeatedly on sub-sampled fluorescence image frames. Specifically, the system can resize the fluorescence image and compare the template image with patches of the resized fluorescence image. In one exemplary implementation, the system can start by comparing the template image against patches (having the same size as the template image) of the original fluorescence image to obtain a first similarity matrix. If there is no similarity score in the first similarity matrix that exceeds the predefined threshold, the system may resize the fluorescence image, and compare the template image against patches (having the same size as the template image) of the resized fluorescence image to obtain a second similarity matrix. The system can iteratively repeat the process until it obtains a similarity score that exceeds the predefined threshold or when the fluorescence image is resized to be the same size as the template image.

After the at least one lymph nodes are identified, the system can display the identified at least one portion of the fluorescence image. For example, the system can indicate the location of the at least one lymph node using a bounding box indicative of the identified at least one portion of the fluorescence image. The identification of the lymph nodes in the fluorescence image can be used to guide a surgical procedure, evaluate a patient, diagnose a disease, recommend a treatment, or any combination thereof. For example, the fluorescence image can be displayed as part of an intraoperative video stream during a surgical procedure, such as a sentinel lymph node mapping procedure.

The system can enhance the visualization of the identified lymph nodes. For example, the system can perform contrast enhancement on the identified at least one portion of the fluorescence image to obtain an enhanced version of the identified at least one portion of the fluorescence image. The contrast enhancement may include localized histogram equalization. To perform localized histogram equalization, the system can apply a histogram equalization algorithm to each region of the image where the similarity values exceed the pre-defined threshold. However, it should be appreciated that other visualization enhancement techniques may be used.

After enhancing the contrast for each identified lymph node, the system may apply a color scheme to further improve the visualization. To visually convey the impression of depth, the system may use the measured average fluorescence intensity of the identified region containing the lymph node as a proxy and apply a color to the region depending on its average intensity value. Specifically, different color schemes can be associated with the different ranges of the average intensity (e.g., red for intensity values between 0-50, orange for 51-101, yellow for 102-152, green for 153 and above). In other words, the system may visualize how dim the original fluorescence signal was, which may be correlated with the depth of the node under the tissue. Accordingly, displaying the enhanced version according to a color scheme may inform the user how deep the lymph node is under the tissue.

Optionally, the color of the lymph node may change dynamically as the lymph node becomes more exposed (and thus its average fluorescence intensity increases) in the process of the surgery. For example, the system may first apply a red color when the original fluorescence signal from the lymph node is very dim. As the surgeon removes the tissue covering the lymph node, the fluorescence signal becomes brighter and the system can switch to a different color (e.g., orange). Finally, when the lymph node is fully exposed, it can be displayed as green.

It should be appreciated that the described visualization technique may not be based on actual depth information. Rather, it may operate under the assumption that the signal intensity is inversely proportional to the node's distance under the tissue surface. There may be other factors that can affect the measured fluorescence intensity, such as ICG dosage. To mitigate this effect, the system may apply normalization factors to the average intensity that take into account, for example, the injected dye dose and the patient's BMI.

FIG. 9 illustrates generation of a template image. The system can generate the template image 902 by extracting the region of a known location of a lymph node from an NIR image 904. By selecting the boundaries of the region of interest, the system can generate a template image that captures the signature spatial distribution of intensities while excluding the patterns that are more common in other types of tissue. For example, as observed in the lymph node intensity distribution plots, the distributions all show a single global peak corresponding to the node's center of mass, with almost symmetrical linear drop of intensities on both side of the maximum. On the other hand, the tail parts of the distributions exhibit more generic patterns and thus have higher likelihood to be found in non-nodal types of fluorescence signal. Accordingly, the system can crop the template image to include the central portion of the node with a small margin on all sides, while excluding the areas towards the edges.

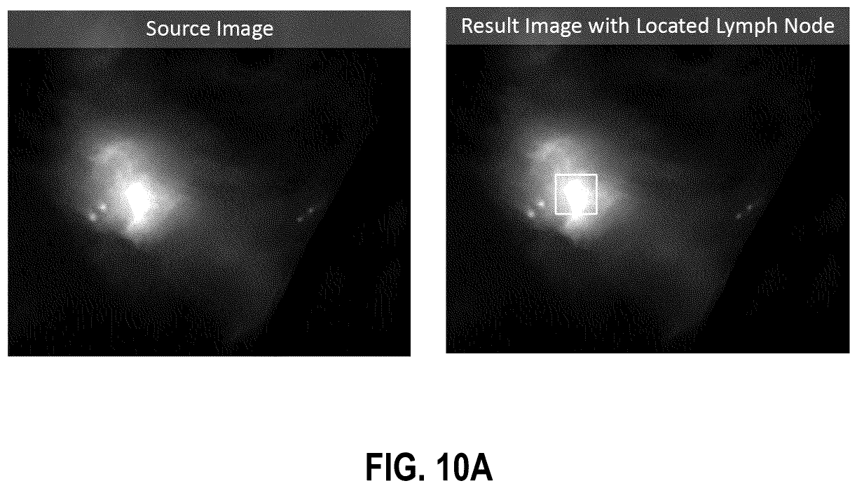

FIGS. 10A-C illustrate exemplary identification of lymph nodes using the techniques described herein. Each figure includes the input fluorescence image and the output image where the lymph node location is outlined by a bounding box. As shown, the techniques described herein can be used to successfully locate the nodes in the NIR frames captured in different modalities and show nodes of various levels of intensities.

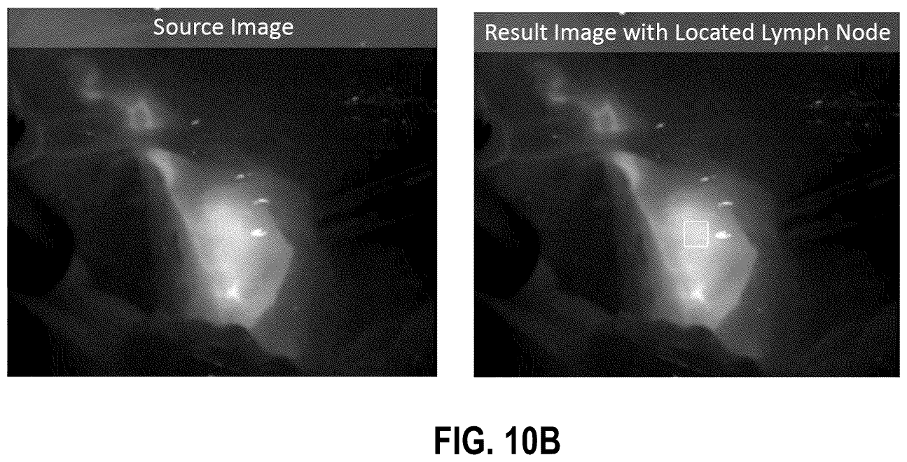

FIG. 11 demonstrates the robustness of the techniques described herein when applied to an extremely dim fluorescence signal. As discussed above, ICG imaging of the lymph nodes usually fails for patients with high BMI because, when a lymph node is buried deep under layers of fatty tissue, the level of its fluorescence signal becomes almost indistinguishable from noise. However, even though the absolute level of the signal is low, its underlying signature of intensity distribution is preserved and thus can allow successful identification by the techniques described herein. In FIG. 11, to emulate the situation with an extremely dim signal generated by a deep lymph node, the signal intensity of an original NIR image 1102 showing an exposed axillary lymph node is divided by a factor of 15 to obtain the dimmed NIR image 1104. The dimmed NIR image 1104 is then processed by the techniques described herein. As shown in the result image 1106, the location of the lymph node is successfully identified.

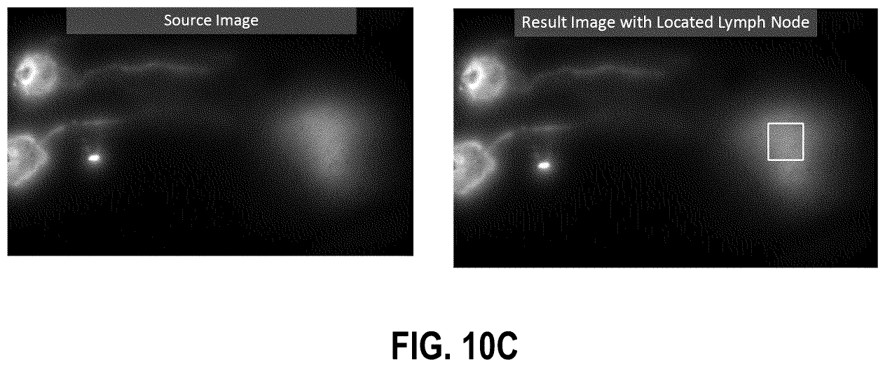

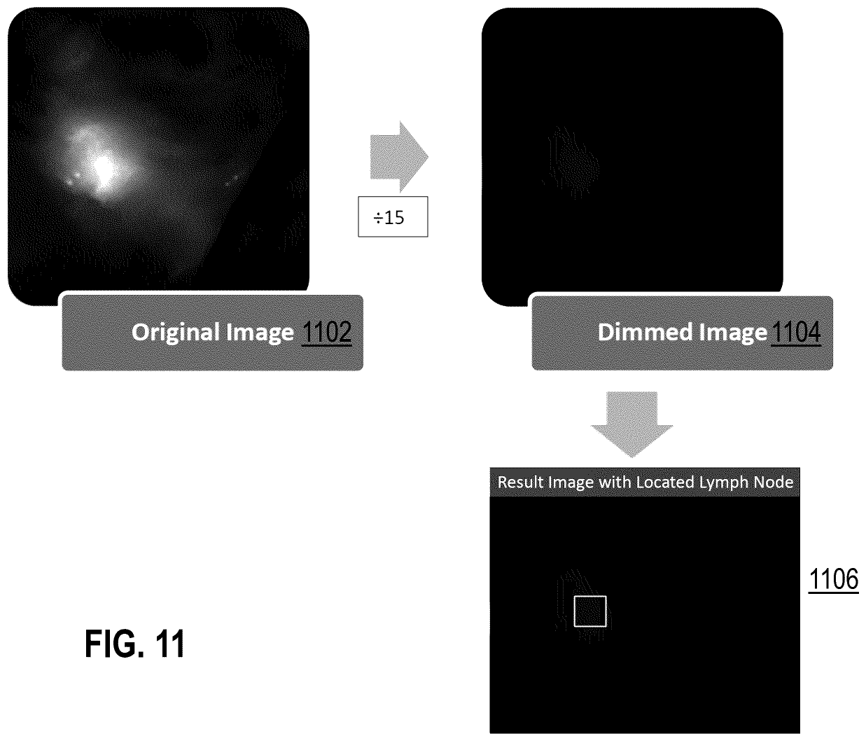

FIG. 12A illustrates a visualization of an exemplary similarity matrix for a fluorescence image. The brightness of the green color of each pixel indicates the amplitude of the corresponding similarity value in the similarity matrix. Accordingly, the brighter a pixel is, the higher the corresponding similarity value is. As shown, the visualization includes one continuous area with the highest brightness level, and some areas that are significantly dimmer. As described herein, a predefined threshold can be applied to eliminate the dim areas such that the one bright area is identified as the location of one or more lymph nodes. FIG. 12B provides an enhanced visualization of the identified lymph node according to a color scheme. By applying localized histogram equalization to the region of interest and then rendering this region of interest in a red-based color scheme, the lymph node is clearly visualized.

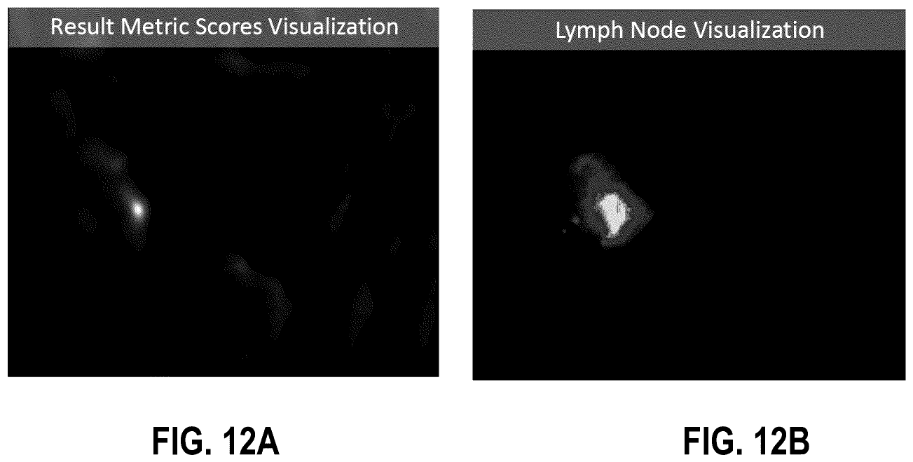

FIG. 13 illustrates an exemplary method 1300 for identifying at least one lymph node of a subject. The method 1300 can automatically recognize the distinct signature of the lymph node intensity distribution in a given NIR input image. Process 1300 is performed, for example, using one or more electronic devices implementing a software platform. In some examples, process 1300 is performed using a client-server system, and the blocks of process 1300 are divided up in any manner between the server and one or more client devices. In some examples, process 1300 is performed using only a client device or only multiple client devices. In process 1300, some blocks are, optionally, combined, the order of some blocks is, optionally, changed, and some blocks are, optionally, omitted. In some examples, additional steps may be performed in combination with the process 1300. Accordingly, the operations as illustrated (and described in greater detail below) are exemplary by nature and, as such, should not be viewed as limiting.

At block 1302, an exemplary system (e.g., one or more electronic devices) obtains a fluorescence image of a field of view including the at least one lymph node of the subject. The fluorescence image may be captured before, during, and/or after a surgical procedure. At block 1304, the system obtains a template image. The template image can be generated using the techniques described herein. As described herein, multiple templates and/or different orientations of the same template may be used.

At block 1306, the system identifies at least one portion of the fluorescence image that corresponds to a location of the at least one lymph node by inputting the template image and the fluorescence image into one or more trained neural networks.

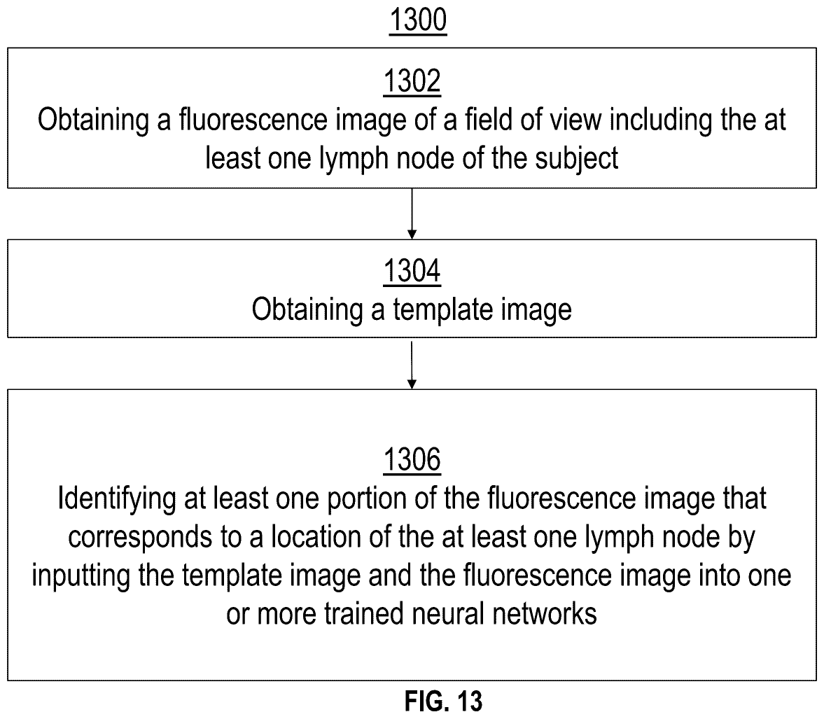

The one or more trained neural networks may comprise an object detection neural network, which can be trained using labeled training images in which lymph nodes are annotated. The annotation may be performed manually or via supervised or self-supervised learning techniques. Optionally, the one or more trained neural networks may comprise deep convolutional neural networks (DNNs) such as object tracking DNNs. The one or more neural networks may comprise a Siamese network backbone, a cross-correlation module connected with the Siamese network backbone, and/or a template localization subnetwork connected with the cross-correlation module. The one or more trained neural networks may be configured to output a classification confidence value for each location of a plurality of locations in the fluorescence image and a template bounding box at the respective location. Additional details of exemplary neural networks are provided below with reference to FIG. 14.

After the at least one lymph nodes are identified, the system can display the identified at least one portion of the fluorescence image and/or enhance the visualization using any of the techniques described herein.

FIG. 14 illustrates an exemplary neural network model. The model can perform the template matching task as a classification-regression task in a number of steps: feature extraction, feature fusion, and template localization. With reference to FIG. 14, the model is configured to receive a template image and a reference image (e.g., a fluorescence image in step 1302 in FIG. 13). The model comprises a Siamese network backbone 1402, a cross-correlation module 1404 connected with the Siamese network backbone, and a template localization subnetwork 1406 connected with the cross-correlation module. The model is configured to output a classification confidence for each location (e.g., whether the location is a part of a lymph node) and the template bounding box at this location. The model may be trained end-to-end on image pairs each comprising a template image and a source image, and their corresponding ground truth classification/regression maps. The classification/regression maps can be generated automatically given the ground-truth bounding boxes outlining the location of the template within the source image. Optionally, the Siamese network backbone 1402 comprises two identical branches with shared parameters, which can be initialized with the weights pre-trained on publicly available datasets. Alternatively, it can be pre-trained in a self-supervised regime on a set of unlabeled fluorescent images containing clinical structures of interest (e.g., lymph nodes). Additional details can be found in Ren et al., “A Robust and Accurate End-to-End Template Matching Method Based on the Siamese Network,” IEEE Geoscience and Remote Sensing Letters, Vol. 19, 2022.

Optionally, in addition to identifying the location of a lymph node using any of the techniques described herein (e.g., FIGS. 8 and 13), the system can also determine the likelihood that the node is metastatic or benign. For example, metastatic nodes tend to have different physical characteristics than reactive or benign lymph nodes. Specifically, some empirical data indicates that metastatic nodes tend to be round with a short to long axes ratio (S/L ratio) greater than 0.5, while reactive or benign lymph nodes are elliptical in shape (S/L ratio <0.5). Accordingly, the system may generate a first template associated with a metastatic lymph node and a second template associated with a reactive or benign lymph node. Each template may be generated by cropping a portion of an actual image and/or based on a known visual shape/pattern, as described herein. The system can compare an image with both the first template and the second template and determine, based on the similarity matrices or machine learning models, whether the identified lymph node is more similar to the first template or the second template. As a result, the system can automatically label the node (e.g., with a metastatic/benign label, with a likelihood of malignancies). Being able to discriminate between normal and metastatic nodes intra-operatively can allow surgeons to better identify the best candidates for biopsy and potentially avoid unnecessary excision of the healthy nodes. Optionally, normal and malignant nodes can be displayed differently. For example, the system can display the nodes using different visual or textual cues, such as different colors for the bounding box, different colors for the identified node, different text labels associated with the identified node (e.g., “malignant—confidence level 78%”), or any combination thereof.

Optionally, the techniques described herein can be used to identify tissue/structure of interest exhibiting a distinct temporal pattern. Certain tissue types (e.g., a specific tumor, a specific lymph node) exhibit distinct temporal patterns of fluorescence dye absorption. For example, a specific type of tumor may absorb the fluorescent dye much faster than healthy tissues. Thus, the system can generate the template image based on a plurality of intensity-over-time curves. Specifically, the system can obtain a time-series sequence of fluorescence images depicting a known tissue of interest over time (e.g., as the fluorescence dye is accumulating, staying, and/or leaving the tissue). The system can crop out the region in each image where the tissue is depicted to obtain a time-series sequence of image patches. Each image location in the time-series sequence is associated with an intensity-over-time curve comprising pixels in the same location across the time-series sequence of images. The template image may be generated based on the plurality of intensity-over-time curves. Specifically, the template image can be generated as a visual map in which the value of each pixel corresponds to a parameter derived from the corresponding intensity-over-time curve for that pixel. The parameter may comprise an ingress rate (i.e., the rate or the slope of the curve at which the intensity of the fluorescence signal is growing), an egress rate (i.e., the rate or the slope of the curve at which the intensity of the fluorescence signal is decreasing), a duration of the stable phase (e.g., the length of the plateau of the curve), or any combination thereof.

After the template image is generated, the system can compare the template image against a given time-series sequence of fluorescence images to detect the tissue of interest. Specifically, the system can calculate an input visual map based on the given time-series sequence of fluorescence images (e.g., by calculating an input visual map of ingress rates) and use the template-matching techniques described herein to compare the template image with the input visual map to identify an area exhibiting a similar visual/temporal pattern as the location of the tissue of interest.

Optionally, the techniques described herein can be used to identify other tissue or anatomical structures of interest exhibiting a distinct visual pattern, such as ureters. Existing techniques for ureter visualization during minimally invasive surgeries include injecting a fluorescent dye (e.g., Methylene Blue (MB) or ICG) and using an NIR camera to visualize the presence of the dye in the ureters. However, there are unique constraints associated with each of the dyes. MB is excreted through the kidneys and is concentrated in the urine. Due to the peristaltic nature of urine movement through the ureters, the latter are only visible periodically when the urine is passing through the ureters. ICG, on the other hand, has a unique property of binding to and staining the proteins of the ureteral epithelium for the entire procedure. However, it requires a cystoscopy-guided ICG instillation to the ureters (vs. a much faster and safer intravenous injection of MB).

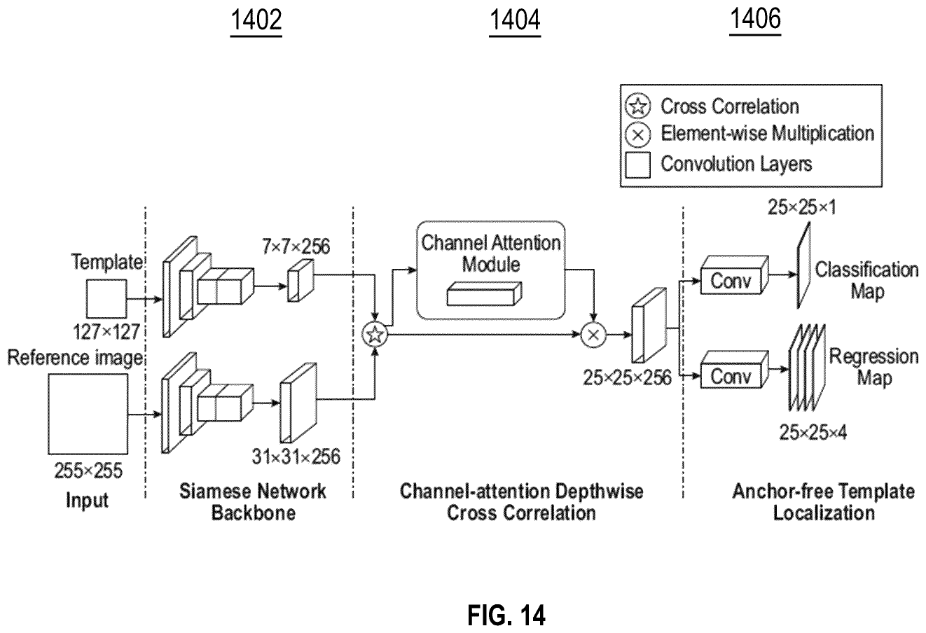

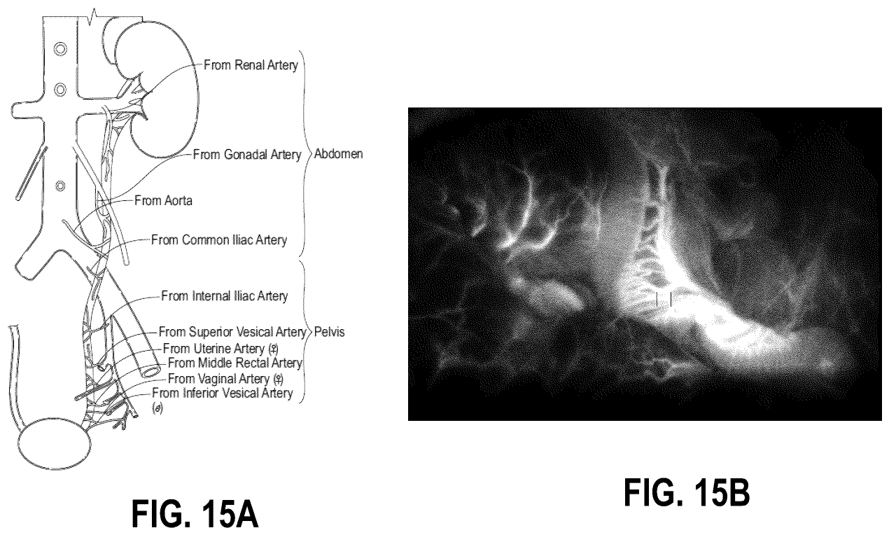

When ICG is administered intravenously, however, it can be detected in blood vessels and is often used for intra-operative perfusion assessment. Accordingly, ureters can be identified due to the unique vasculature network surrounding them. FIG. 15A illustrates an anatomical diagram of a ureter vasculature system and FIG. 15B illustrates its appearance in the fluorescent mode. As shown in FIG. 15A, each ureter is densely wrapped by a spiral network of vessels. This pattern is also clearly visible in the NIR image in FIG. 15B and is different from other fluorescing vessels. Hence, using the techniques described herein, the system can generate template images of ICG-fluorescing ureters and then use them to locate ureters in NIR frames acquired during a surgery.

The foregoing description, for the purpose of explanation, has been described with reference to specific examples or aspects. However, the illustrative discussions above are not intended to be exhaustive or to limit the invention to the precise forms disclosed. For the purpose of clarity and a concise description, features are described herein as part of the same or separate variations; however, it will be appreciated that the scope of the disclosure includes variations having combinations of all or some of the features described. Many modifications and variations are possible in view of the above teachings. The variations were chosen and described in order to best explain the principles of the techniques and their practical applications. Others skilled in the art are thereby enabled to best utilize the techniques and various variations with various modifications as are suited to the particular use contemplated.

Although the disclosure and examples have been fully described with reference to the accompanying figures, it is to be noted that various changes and modifications will become apparent to those skilled in the art. Such changes and modifications are to be understood as being included within the scope of the disclosure and examples as defined by the claims. Finally, the entire disclosure of the patents and publications referred to in this application are hereby incorporated herein by reference.

What is claimed is:

1. A method for identifying at least one lymph node of a subject, comprising:
obtaining a fluorescence image of a field of view including the at least one lymph node of the subject;
obtaining a template image;
comparing the template image with the fluorescence image to obtain one or more similarity values; and
identifying at least one portion of the fluorescence image that corresponds to a location of the at least one lymph node based on the one or more similarity values.

2. The method of claim 1, further comprising: displaying the identified at least one portion of the fluorescence image.

3. The method of claim 2, wherein displaying the identified at least one portion of the fluorescence image comprises: displaying the fluorescence image and at least one bounding box indicative of the identified at least one portion of the fluorescence image.

4. The method of claim 2, wherein the fluorescence image is displayed as part of an intraoperative video stream during a surgical procedure.

5. The method of claim 4, wherein the surgical procedure comprises a sentinel lymph node mapping procedure.

6. The method of claim 1, wherein the fluorescence image is a first fluorescence image, wherein the field of view is a first field of view, and wherein the subject is a first subject, the method further comprising:
obtaining a second fluorescence image of a second field of view including a lymph node of a second subject different from the first subject; and
cropping a portion of the second fluorescence image as the template image.

7. The method of claim 6, wherein the cropped portion of the second fluorescence image depicts a center portion of the lymph node of the second subject.

8. The method of claim 1, wherein the template image is generated based on a predefined intensity distribution pattern.

9. The method of claim 1, wherein the template image is generated based on a plurality of candidate template images.

10. The method of claim 9, wherein the plurality of candidate template images corresponds to a plurality of subjects.

11. The method of claim 1, wherein comparing the template image with the fluorescence image comprises:
comparing the template image with a plurality of patches of the fluorescence image; and
generating a matrix of similarity values, each similarity value in the matrix indicative of a difference between the template image and a respective patch of the plurality of patches of the fluorescence image.

12. The method of claim 11, wherein each similarity value in the matrix of similarity values is calculated based on a pixel-wise comparison between the template image and the respective patch of the plurality of patches of the fluorescence image.

13. The method of claim 11, wherein the plurality of patches of the fluorescence image is a first plurality of patches of the fluorescence image, the method further comprising:
resizing the fluorescence image; and
comparing the template image with a second plurality of patches of the resized fluorescence image.

14. The method of claim 1, wherein identifying the at least one portion of the fluorescence image corresponding to the at least one lymph node comprises: comparing each similarity value of the one or more similarity values with a predefined threshold.

15. The method of claim 1, further comprising: performing contrast enhancement on the identified at least one portion of the fluorescence image to obtain an enhanced version of the identified at least one portion of the fluorescence image.

16. The method of claim 15, further comprising: displaying the enhanced version of the identified at least one portion of the fluorescence image according to a color scheme.

17. The method of claim 15, further comprising: wherein the contrast enhancement comprises histogram equalization.

18. The method of claim 1, wherein the template image is associated with a metastatic node, the method further comprising:
determining whether the at least one lymph node is metastatic based on the one or more similarity values.

19. The method of claim 18, further comprising: displaying a visual indication of the metastatic determination for each lymph node of the at least one lymph node.

20. The method of claim 1, wherein the template image is generated based on a plurality of time-intensity curves.

21. The method of claim 20, wherein the fluorescence image is generated based on a time series of signal intensity data.

22. A system for identifying at least one lymph node of a subject, comprising:
one or more processors;
one or more memories; and
one or more programs, wherein the one or more programs are stored in the one or more memories and configured to be executed by the one or more processors, the one or more programs including instructions for:
obtaining a fluorescence image of a field of view including the at least one lymph node of the subject;
obtaining a template image;
comparing the template image with the fluorescence image to obtain one or more similarity values; and
identifying at least one portion of the fluorescence image that corresponds to a location of the at least one lymph node based on the one or more similarity values.

23. A non-transitory computer-readable storage medium storing one or more programs for identifying at least one lymph node of a subject, the one or more programs comprising instructions, which when executed by one or more processors of an electronic device, cause the electronic device to perform:
obtaining a fluorescence image of a field of view including the at least one lymph node of the subject;
obtaining a template image;
comparing the template image with the fluorescence image to obtain one or more similarity values; and
identifying at least one portion of the fluorescence image that corresponds to a location of the at least one lymph node based on the one or more similarity values.
